(19) United States
(12) Patent Application Publication
(10) Pub. No.: US 2022/0222535 A1
(43) Pub. Date: Jul. 14, 2022

(54) COMPUTING DEVICE AND METHOD USING A NEURAL NETWORK TO ANALYZE TEMPERATURE MEASUREMENTS OF AN INFRARED SENSOR

(71) Applicant: Distech Controls Inc., Brossard (CA)

(72) Inventors: Francois GERVAIS, Lachine (CA); Jean-Simon BOUCHER, Sainte-Julie (CA)

(21) Appl. No.: 17/144,744

(22) Filed: Jan. 8, 2021

(51) Int. Cl. G06N 3/08; G06K 9/62; G06F 9/30; A61B 5/00

(52) U.S. Cl. CPC G06N 3/084; A61B 5/0008; G06F 9/30021; G06K 9/6256

(57) ABSTRACT

Method and computing device using a neural network to analyze temperature measurements of an infrared sensor. A predictive model of the neural network is stored by the computing device. The computing device receives a two-dimensional (2D) matrix of temperature measurements generated by the IR sensor, and executes the neural network using the predictive model for generating outputs based on inputs. The inputs comprise the 2D matrix of temperature measurements. The outputs comprise a 2D matrix of inferred temperatures. The computing device determines a subset of values of the 2D matrix of temperature measurements, and applies a comparison algorithm to the subset of values of the 2D matrix of temperature measurements and a corresponding subset of values of the 2D matrix of inferred temperatures, to detect an anomaly in the subset of values of the 2D matrix of temperature measurements. A method for training the neural network is also provided.

### TECHNICAL FIELD

The present disclosure relates to the field of infrared sensors. More specifically, the present disclosure presents a computing device and method using a neural network to analyze temperature measurements of an infrared sensor.

### BACKGROUND

Systems for controlling environmental conditions, for example in buildings, are becoming increasingly sophisticated. An environment control system may at once control heating and cooling, monitor air quality, detect hazardous conditions such as fire, carbon monoxide release, intrusion, and the like. Such environment control systems generally include at least one environment controller, which receives measured environmental values, generally from sensors, and in turn determines set-points or command parameters to be sent to controlled appliances.

One type of sensor used in the context of environment control systems is an infrared (IR) sensor, such as for example an IR camera. A commonly used output of the IR sensor is a temperature measurement, or a two-dimensional (2D) matrix of temperature measurements. The temperature measurement(s) generated by the IR sensor may have different applications, such as detecting the presence of a person in an area, counting the number of persons present in an area, evaluating the body temperature of a person present in the area, evaluating the external temperature of an object located in the area, etc. The temperature measurement(s) performed by the IR sensor is used, generally in combination with data from other sensors, by an environment controller for controlling operations of one or more controlled appliance.

The present disclosure focuses on IR cameras (or any other type of IR sensor) used to measure the body temperature of a human being, in order to detect a medical condition such as fever. The IR camera is generally operated by an attendant, who points the IR camera to the person being monitored (e.g. towards the head of the monitored person). The attendant analyzes the temperature measurement(s) generated by the IR camera, to determine whether the monitored person has a medical condition.

Relying on an attendant for operating the IR camera and performing the temperature(s) analysis has several drawbacks. For instance, the analysis of the temperature(s) performed by the attendant is subject to error and bias. Furthermore, the efficiency of the procedure is limited, and could be improved through automation.

Therefore, there is a need for a computing device and method using a neural network to analyze temperature measurements of an infrared sensor.

### SUMMARY

According to a first aspect, the present disclosure relates to a computing device. The computing device comprises a communication interface, memory, and a processing unit comprising one or more processor. The memory stories a predictive model generated by a neural network training engine. The processing unit receives via the communication interface a two-dimensional (2D) matrix of temperature measurements from an infrared (IR) sensor. The processing unit executes a neural network inference engine. The neural network inference engine implements a neural network using the predictive model for generating outputs based on inputs. The inputs comprise the 2D matrix of temperature measurements. The outputs comprise a 2D matrix of inferred temperatures. The processing unit determines a subset of values of the 2D matrix of temperature measurements. The processing unit applies a comparison algorithm to the subset of values of the 2D matrix of temperature measurements and a corresponding subset of values of the 2D matrix of inferred temperatures.

According to a second aspect, the present disclosure relates to a method using a neural network to analyze temperature measurements of an infrared sensor. The method comprises storing a predictive model generated by a neural network training engine in a memory of a computing device. The method comprises receiving by the processing unit of the computing device a two-dimensional (2D) matrix of temperature measurements generated by the IR sensor. The method comprises executing by the processing unit of the computing device a neural network inference engine. The neural network inference engine implements a neural network using the predictive model for generating outputs based on inputs. The inputs comprise the 2D matrix of temperature measurements. The outputs comprise a 2D matrix of inferred temperatures. The method comprises determining by the processing unit of the computing device a subset of values of the 2D matrix of temperature measurements. The method comprises applying by the processing unit of the computing device a comparison algorithm to the subset of values of the 2D matrix of temperature measurements and a corresponding subset of values of the 2D matrix of inferred temperatures.

In a particular aspect, the processing unit of the computing device determines that the subset of values of the 2D matrix of temperature measurements is anomalous based on a result of the comparison algorithm.

In another particular aspect, the 2D matrix of temperature measurements comprises body temperature measurements of a human being, the subset of values of the 2D matrix of temperature measurements comprises at least some of the body temperature measurements of the human being, and the determination that the subset of values of the 2D matrix of temperature measurements is anomalous is indicative of the human being having fever.

According to a third aspect, the present disclosure relates to a method for training a neural network to analyze temperature measurements of an infrared (IR) sensor. The method comprises (a) initializing by a processing unit of a computing device a predictive model of a neural network. The method comprises (b) generating by the processing unit of the computing device training data, the training data comprising a plurality of two-dimensional (2D) matrices of temperature measurements generated by an IR sensor. The method comprises (c) executing by the processing unit of the computing device a neural network training engine. The neural network training engine implements the neural network using the predictive model for generating outputs based on inputs. The outputs comprise a 2D matrix of inferred temperatures. The inputs comprise a given 2D matrix of temperature measurements among the plurality of 2D matrices of temperature measurements. The method comprises (d) adjusting by the processing unit of the computing device the predictive model of the neural network to minimize a difference between the 2D matrix of inferred temperatures and the given 2D matrix of temperature measurements.

In a particular aspect. steps (c) and (d) are repeated for several 2D matrices of temperature measurements among the plurality of 2D matrices of temperature measurements.

### BRIEF DESCRIPTION OF THE DRAWINGS

Embodiments of the disclosure will be described by way of example only with reference to the accompanying drawings, in which:

FIG. 1 illustrates an environment control system comprising a computing device and infrared (IR) sensor;

FIG. 2 provides a detailed representation of the IR sensor of FIG. 1;

FIG. 3 provides a detailed representation of a training server represented in FIG. 1;

FIG. 4 illustrates a method implemented by the computing device of FIG. 1, using a neural network to analyze temperature measurements of the IR sensor of FIG. 1;

FIG. 5 is a schematic representation of a neural network inference engine executed by the computing device of FIG. 1 according to the method of FIG. 4;

FIGS. 6A and 6B illustrate exemplary implementations of a neural network implemented by the neural network inference engine of FIG. 5; and

FIG. 7 illustrates a method implemented by the training server of FIG. 1, for training a neural network to analyze temperature measurements of the IR sensor of FIG. 1.

### DETAILED DESCRIPTION

The foregoing and other features will become more apparent upon reading of the following non-restrictive description of illustrative embodiments thereof, given by way of example only with reference to the accompanying drawings.

Various aspects of the present disclosure generally address one or more of the problems related to environment control systems for buildings. More particularly, the present disclosure aims at providing solutions for analyzing temperature measurements performed by an infrared (IR) sensor, to detect anomalies in the temperature measurements. For example, the IR sensor performs body temperature measurements of a human being and the anomalies in the body temperature measurements are indicative of the human being having fever.

The following terminology is used throughout the present specification:

Environment: condition(s) (temperature, pressure, oxygen level, light level, security, etc.) prevailing in a controlled area or place, such as for example in a building.

Environment control system: a set of components which collaborate for monitoring and controlling an environment.

Environmental data: any data (e.g. information, commands) related to an environment that may be exchanged between components of an environment control system.

Environment control device (ECD): generic name for a component of an environment control system. An ECD may consist of an environment controller, a sensor, a controlled appliance, etc.

Environment controller: device capable of receiving information related to an environment and sending commands based on such information.

Environmental characteristic: measurable, quantifiable or verifiable property of an environment (a building). The environmental characteristic comprises any of the following: temperature, pressure, humidity, lighting, CO2, flow, radiation, water level, speed, sound; a variation of at least one of the following, temperature, pressure, humidity and lighting, CO2 levels, flows, radiations, water levels, speed, sound levels, etc., and/or a combination thereof.

Environmental characteristic value: numerical, qualitative or verifiable representation of an environmental characteristic.

Sensor: device that detects an environmental characteristic and provides a numerical, quantitative or verifiable representation thereof. The numerical, quantitative or verifiable representation may be sent to an environment controller.

Controlled appliance: device that receives a command and executes the command. The command may be received from an environment controller.

Environmental state: a current condition of an environment based on an environmental characteristic, each environmental state may comprise a range of values or verifiable representation for the corresponding environmental characteristic.

VAV appliance: a Variable Air Volume appliance is a type of heating, ventilating, and/or air-conditioning (HVAC) system. By contrast to a Constant Air Volume (CAV) appliance, which supplies a constant airflow at a variable temperature, a VAV appliance varies the airflow at a constant temperature.

Area of a building: the expression ‘area of a building’ is used throughout the present specification to refer to the interior of a whole building or a portion of the interior of the building such as, without limitation: a floor, a room, an aisle, etc.

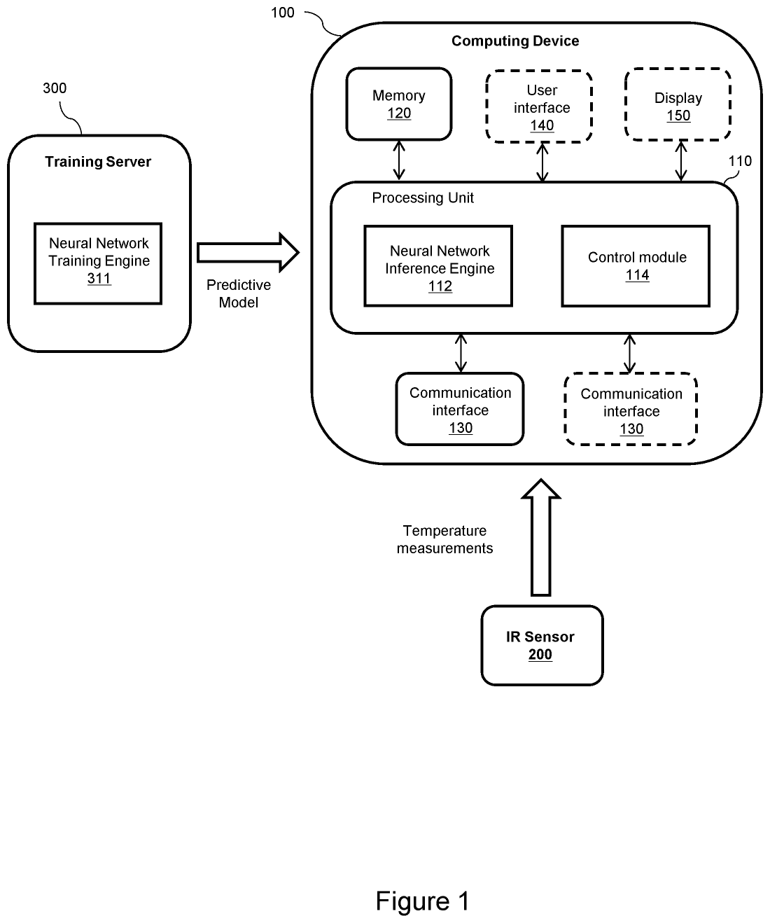

Referring now to FIGS. 1, 2 and 3, an environment control system comprising a computing device 100 is illustrated. For example, the computing device 100 is an environment controller 100 exchanging data with other environment control devices (ECDs). The environment controller 100 is responsible for controlling the environment of an area of a building. The environment controller 100 receives from sensors (e.g. from an infrared (IR) sensor 200) environmental characteristic values measured by the sensors. The environment controller 100 generates commands based on the received environmental characteristic values. The generated commands are transmitted to controlled appliances (not represented in the Figures for simplification purposes), to control operations of the controlled appliances.

The area under the control of the computing device 100 is not represented in the Figures for simplification purposes. As mentioned previously, the area may consist of a room, a floor, an aisle, etc. However, any type of area located inside any type of building is considered being within the scope of the present disclosure.

Details of the computing device 100, the IR sensor 200 and the training server 300 will now be provided.

As illustrated in FIG. 1, the computing device 100 comprises a processing unit 110, memory 120, and a communication interface 130. The computing device 100 may comprise additional components, such as another communication interface 130, a user interface 140, a display 150, etc.

The processing unit 110 comprises one or more processor (not represented in the Figures) capable of executing instructions of a computer program. Each processor may further comprise one or several cores. The processing unit 110 executes a neural network inference engine 112 and a control module 114, as will be detailed later in the description.

The memory 120 stores instructions of computer program(s) executed by the processing unit 110 (for implementing the neural network inference engine 112, the control module 114, etc.), data generated by the execution of the computer program(s), data received via the communication interface 130 (or another communication interface), etc. Only a single memory 120 is represented in FIG. 1, but the environment controller 100 may comprise several types of memories, including volatile memory (such as a volatile Random Access Memory (RAM), etc.) and non-volatile memory (such as a hard drive, electrically-erasable programmable read-only memory (EEPROM), flash, etc.).

The communication interface 130 allows the computing device 100 to exchange data with remote devices (e.g. with the IR sensor 200, controlled appliance(s), a training server 300, etc.) over a communication network (not represented in FIG. 1 for simplification purposes). For example, the communication network is a wired communication network, such as an Ethernet network; and the communication interface 130 is adapted to support communication protocols used to exchange data over the Ethernet network. Other types of wired communication networks may also be supported by the communication interface 130. In another example, the communication network is a wireless communication network, such as a Wi-Fi network; and the communication interface 130 is adapted to support communication protocols used to exchange data over the Wi-Fi network. Other types of wireless communication network may also be supported by the communication interface 130, such as a wireless mesh network, Bluetooth®, Bluetooth® Low Energy (BLE), etc. In still another example, the environment controller 100 comprises two communication interfaces 130. The environment controller 100 communicates with the IR sensor 200 and controlled appliance(s) via a first communication interface 130 (e.g. a Wi-Fi interface); and communicates with other devices (e.g. the training server 300) via a second communication interface 130 (e.g. an Ethernet interface). Each communication interface 130 usually comprises a combination of hardware and software executed by the hardware, for implementing the communication functionalities of the communication interface 130.

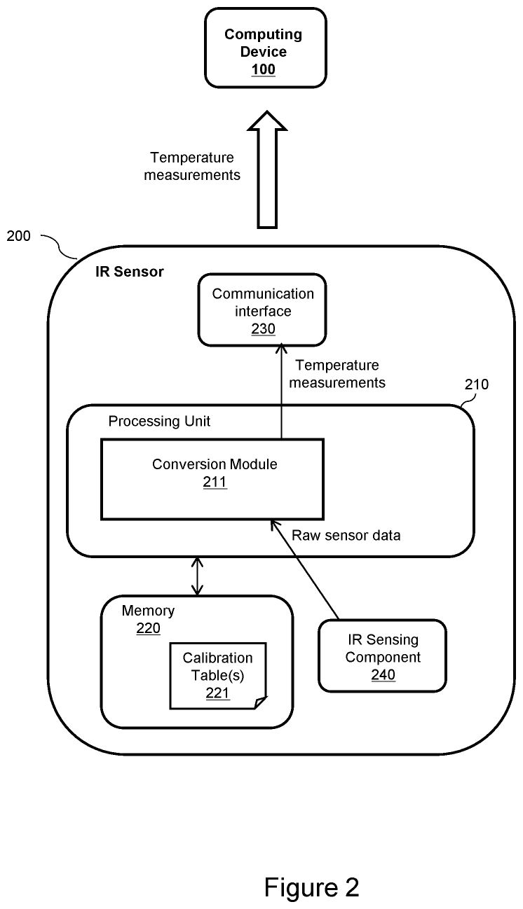

As illustrated in FIG. 2, the IR sensor 200 comprises a processing unit 210, memory 220, a communication interface 230 and an IR sensing component 240. The IR sensor 200 may comprise additional components not represented in the Figures. An example of IR sensor 200 consists of an IR camera. However, other types of IR sensor may be used in place of an IR camera.

The processing unit 210 of the IR sensor 200 is similar to the processing unit 110 of the computing device 100. The processing unit 210 executes a conversion module 211, as will be detailed later in the description.

The memory 220 of the IR sensor 200 is similar to the memory 120 of the computing device 100. The memory 220 stores instructions of computer program(s) executed by the processing unit 210 for implementing the conversion module 211, etc. The memory 220 also stores calibration table(s) 221, which will be detailed later in the description.

The communication interface 230 of the IR sensor 200 is similar to the communication 130 of the computing device 100. The communication interface 230 allows the IR sensor 200 to exchange data with remote devices (e.g. with the computing device 100, etc.) over a communication network (not represented in FIG. 2 for simplification purposes). The communication interface 230 is generally a wireless communication interface, such as a Wi-Fi interface.

The IR sensing component 240 is well known in the art of IR sensors. The IR sensing component 240 generates raw sensor data. The conversion module 211 executed by the processing unit 210 is a software implementing a conversion algorithm. The conversion algorithm uses the calibration table(s) 221 to convert the raw sensor data into corresponding temperature measurements. The raw sensor data may be pre-processed, before processing by the conversion algorithm.

For example, in the case of a low end IR camera, the IR sensing component 240 performs thermographic IR imaging, which consists in detecting radiations in the electromagnetic spectrum with wavelengths in the range of substantially 900 to 14 000 nanometers. The IR sensing component 240 implements a thermal detector which collects heat energy. The thermal detector produces a signal voltage proportional to the collected heat energy. The signal voltage is converted into a digital count by an Analog to Digital (A/D) converter. The A/D converter (not represented in FIG. 2 for simplification purposes) may be integrated to the IR sensing component 240 or to the processing unit 210. In this exemplary implementation, the raw sensor data represented in FIG. 2 consist of the digital counts, which are integers encoded on a given number of bits (e.g. 14 bits providing a range of values of 0 to 16383). The conversion algorithm converts each digital count into a corresponding temperature measurement, using the calibration table(s) 221. The details of how the conversion algorithm and the calibration table(s) 221 are implemented are out of the scope of the present disclosure. The conversion algorithm generally operates in two steps. In a first step, the digital count is converted into a radiance value. In a second step, the radiance value is converted into the temperature measurement, using a known or measured emissivity value of the target object (or person) which temperature is measured. FIG. 2 illustrates the aforementioned process, where the conversion module 211 converts the raw sensor data into temperature measurements, which are transmitted to the computing device 100.

As mentioned previously, the present disclosure addresses IR sensors 200 which generate a two-dimensional (2D) matrix of temperature measurements. The conversion module 211 receives a 2-D matrix of raw sensor data, comprising M lines and N columns (for example, M is equal to 24 and N is equal to 32), as follows:

[R_{1,1} R₁,2 . . . R_{1,M}

R_{2,1} R_{2,2} . . . R_{2,M}

. . .

R_{N,1} R_{N,2} . . . R_{N,M}]

The conversion module 211 generates a 2-D matrix of temperature measurements, comprising M lines and N columns, as follows:

[T_{1,1} T_{1,2} . . . T_{1,M}

T_{2,1} T_{2,2} . . . T_{2,M}

. . .

T_{N,1} T_{N,2} . . . T_{N,M}]

Each value of the matrix of raw sensor data is representative of heat energy collected by the IR sensing component 240. Each corresponding value T_{i,j} of the matrix of temperature measurements is calculated by applying the calibration table(s) 221 to the value of R_{i,j}.

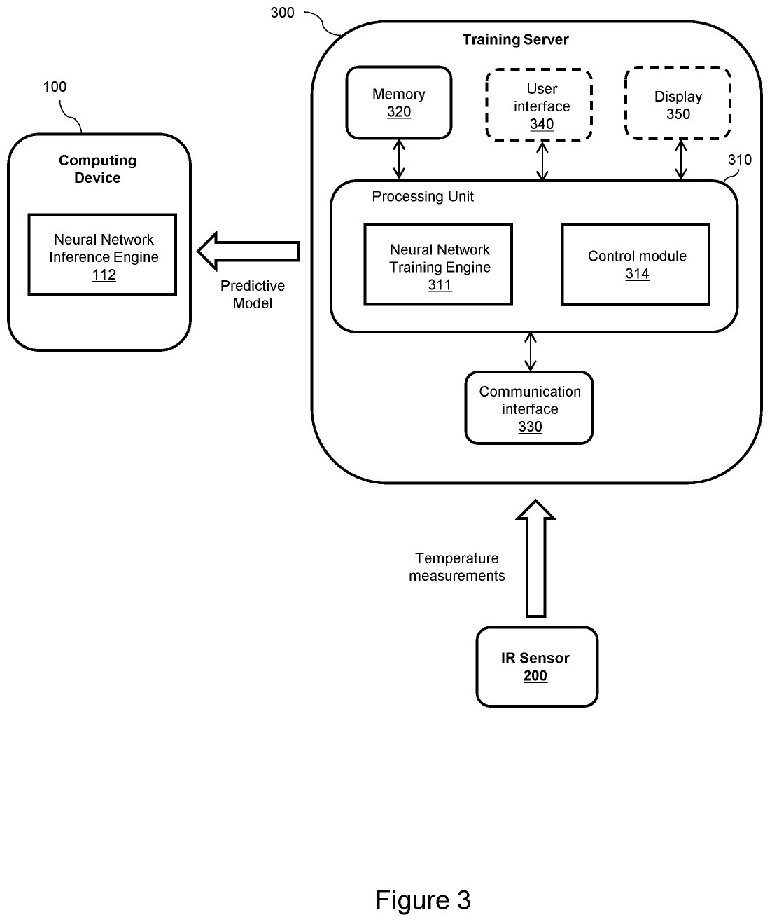

As illustrated in FIG. 3, the training server 300 comprises a processing unit 310, memory 320 and a communication interface 330. The training server 300 may comprise additional components, such as a user interface 340, a display 350, etc.

The processing unit 310 of the training server 300 is similar to the processing unit 110 of the computing device 100. The processing unit 310 executes a neural network training engine 311 and a control module 314, as will be detailed later in the description.

The memory 320 of the training server 300 is similar to the memory 120 of the computing device 100. The memory 320 stores instructions of computer program(s) executed by the processing unit 310 for implementing the neural network training engine 311, the control module 314, etc.

The communication interface 330 of the training server 300 is similar to the communication 130 of the computing device 100. The communication interface 330 allows the training server 300 to exchange data with remote devices (e.g. with the computing device 100, etc.) over a communication network (not represented in FIG. 3 for simplification purposes).

The execution of the neural network training engine 311 generates a predictive model of a neural network, which is transmitted to the computing device 100 via the communication interface of the training server 300. The predictive model is transmitted over a communication network and received via the communication interface 130 of the computing device 100.

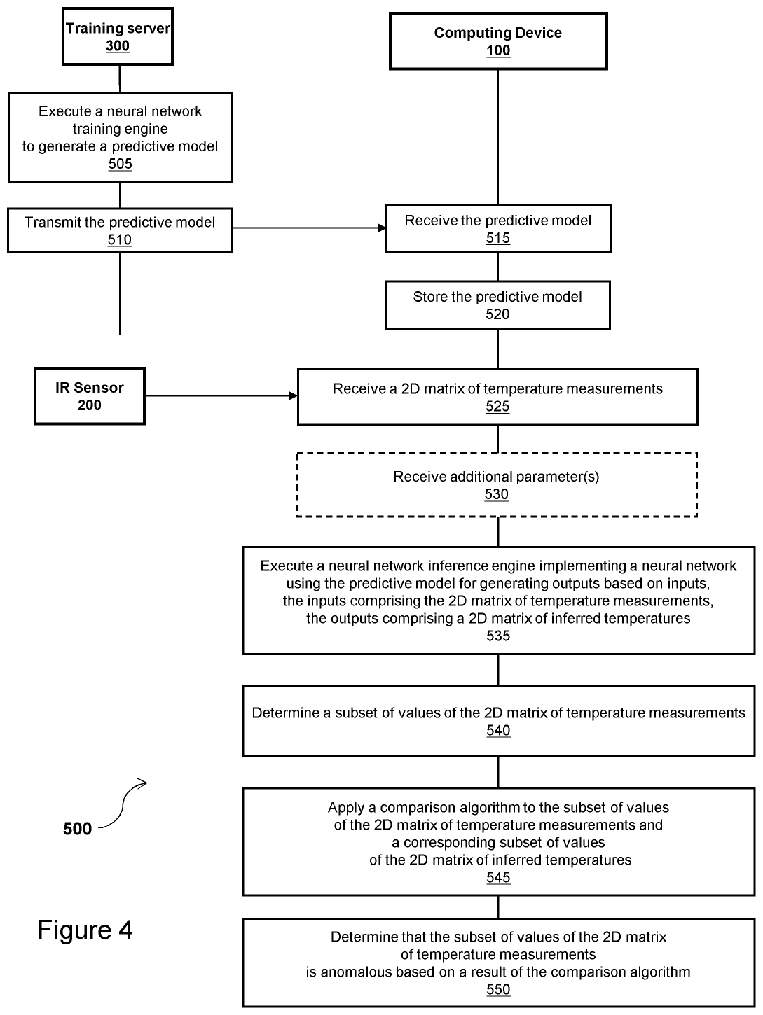

Reference is now made concurrently to FIGS. 1, 2, 3 and 4; where FIG. 4 represents a method 500 using a neural network to analyze temperature measurements of an IR sensor. At least some of the steps of the method 500 are implemented by the computing device 100. The present disclosure is not limited to the method 500 being implemented by the computing device 100 represented in FIG. 1, but is applicable to any type of computing device capable of implementing the steps of the method 500.

A dedicated computer program has instructions for implementing at least some of the steps of the method 500. The instructions are comprised in a non-transitory computer program product (e.g. the memory 120) of the computing device 100. The instructions provide for using a neural network to analyze temperature measurements of an IR sensor, when executed by the processing unit 110 of the computing device 100. The instructions are deliverable to the computing device 100 via an electronically-readable media such as a storage media (e.g. CD-ROM, USB key, etc.), or via communication links (e.g. via a communication network through the communication interface 130).

The instructions of the dedicated computer program executed by the processing unit 110 implement the neural network inference engine 112 and the control module 114. The neural network inference engine 112 provides functionalities of a neural network, allowing to infer output(s) based on inputs using the predictive model (generated by the training server 300), as is well known in the art. The control module 114 provides functionalities allowing the computing device 100 to interact with and control other devices (e.g. the IR sensor 200 and the training server 300).

The method 500 comprises the step 505 of executing the neural network training engine 311 to generate the predictive model. Step 505 is performed by the processing unit 310 of the training server 300. The predictive model comprises parameters of a neural network implemented by the neural network training engine 311. This step will be further detailed later in the description.

The method 500 comprises the step 510 of transmitting the predictive model generated at step 505 to the computing device 100, via the communication interface 330 of the training server 300. Step 510 is performed by the processing unit 310 of the training server 300.

The method 500 comprises the step 515 of receiving the predictive model from the training server 300, via the communication interface 130 of the computing device 100. Step 515 is performed by the processing unit 110 of the computing device 100.

The method 500 comprises the step 520 of storing the predictive model in the memory 120 of the computing device 100. Step 520 is performed by the processing unit 110 of the computing device 100.

The method 500 comprises the step 525 of receiving a two-dimensional matrix of temperature measurements from the IR sensor 200, via the communication interface 130 of the computing device 100. Step 525 is performed by the processing unit 110 of the computing device 100.

The method 500 comprises the optional step 530 of receiving additional parameter(s), via the communication interface 130 of the computing device 100. Step 530 is performed by the processing unit 110 of the computing device 100.

The method 500 comprises the step 535 of executing the neural network inference engine 112. The neural network inference engine 112 implements a neural network using the predictive model (stored at step 520) for generating outputs based on inputs. The execution of the neural network inference engine 112 is performed by the processing unit 110 of the computing device 100. This step will be further detailed later in the description.

The inputs comprise the two-dimensional matrix of temperature measurements received at step 525. Optionally, the inputs also include the additional parameter(s) received at step 530. During the training phase, the predictive model generated by the neural network training engine 311 takes into account the additional parameter(s) if applicable.

The outputs comprise a two-dimensional matrix of inferred temperatures.

The method 500 comprises the step 540 of determining a subset of values of the 2D matrix of temperature measurements (received at step 525). Step 540 is performed by the processing unit 110 of the computing device 100.

In an exemplary implementation, the subset of values is determined by the processing unit 110 based on data received via at least one of the communication interface 130 and the user interface 140. The data identify positions in the 2D matrix of temperature measurements, and the subset of values consists of the values corresponding to the identified positions.

The method 500 comprises the step 545 of applying a comparison algorithm to the subset of values of the 2D matrix of temperature measurements (determined at step 540) and a corresponding subset of values of the 2D matrix of inferred temperatures. Step 545 is performed by the processing unit 110 of the computing device 100.

The method 500 comprises the step 550 of determining that the subset of values of the 2D matrix of temperature measurements is anomalous based on a result of the comparison algorithm executed at step 545. Step 550 is performed by the processing unit 110 of the computing device 100. The determination performed at step 550 depends on the implementation of the comparison algorithm at step 545.

In a first exemplary implementation of step 545, the comparison algorithm calculates an average of the absolute differences between the corresponding values of the two subsets, which is equal to the sum of the absolute values of the differences between each value of the subset of values of the 2D matrix of temperature measurements and each corresponding value of the subset of values of the 2D matrix of inferred temperatures, divided by the number of values in the subsets. At step 550, the averaged absolute difference calculated by the comparison algorithm is compared to a pre-defined threshold, to determine if the subset of values of the 2D matrix of temperature measurements is anomalous (e.g. the averaged absolute difference is greater or equal than the threshold) or normal (e.g. the averaged absolute difference is lower than the threshold).

In a second exemplary implementation of step 545, the comparison algorithm calculates the means square error (MSE) of the subset of values of the 2D matrix of temperature measurements and the corresponding subset of values of the 2D matrix of inferred temperatures. The calculation of the MSE of two sets of data is well known in the art. At step 550, the MSE calculated by the comparison algorithm is compared to a pre-defined threshold, to determine if the subset of values of the 2D matrix of temperature measurements is anomalous (e.g. the MSE is greater or equal than the threshold) or normal (e.g. the MSE is lower than the threshold).

In a third exemplary implementation of step 545, the comparison algorithm calculates the number of occurrences for which the absolute difference between a given value of the subset of values of the 2D matrix of temperature measurements and the corresponding given value of the subset of values of the 2D matrix of inferred temperatures is greater than a pre-defined difference value. At step 550, the number of occurrences calculated by the comparison algorithm is compared to a pre-defined threshold, to determine if the subset of values of the 2D matrix of temperature measurements is anomalous (e.g. the number of occurrences is greater or equal than the threshold) or normal (e.g. the number of occurrences is lower than the threshold). The threshold can be expressed as a percentage, by dividing the number of occurrences by the number of values of the subset of values of the 2D matrix of temperature measurements.

The aforementioned exemplary implementations of the comparison algorithms are for illustration purposes only. A person skilled in the art would readily understand that other implementations of the comparison algorithm may be used at step 545.

Following is an exemplary application of the method 500 to a 2D matrix having 6 lines and 5 columns. The number of lines (6) and columns (5) of the 2D matrix are for illustration purposes only. A person skilled in the art would readily apply the following to a matrix having different dimensions.

The 2D matrix of temperature measurements is as follows:

| | | | | | |
|---|---|---|---|---|---|
| T_{1,1} | T_{1,2} | T_{1,3} | T_{1,4} | T_{1,5} | T_{1,6} |
| T_{2,1} | T_{2,2} | T_{2,3} | T_{2,4} | T_{2,5} | T_{2,6} |
| T_{3,1} | T_{3,2} | T_{3,3} | T_{3,4} | T_{3,5} | T_{3,6} |
| T_{4,1} | T_{4,2} | T_{4,3} | T_{4,4} | T_{4,5} | T_{4,6} |
| T_{5,1} | T_{5,2} | T_{5,3} | T_{5,4} | T_{5,5} | T_{5,6} |

The 2D matrix of inferred temperatures is as follows:

| | | | | | |
|---|---|---|---|---|---|
| T′_{1,1} | T′_{1,2} | T′_{1,3} | T′_{1,4} | T′_{1,5} | T′_{1,6} |
| T′_{2,1} | T′_{2,2} | T′_{2,3} | T′_{2,4} | T′_{2,5} | T′_{2,6} |
| T′_{3,1} | T′_{3,2} | T′_{3,3} | T′_{3,4} | T′_{3,5} | T′_{3,6} |
| T′_{4,1} | T′_{4,2} | T′_{4,3} | T′_{4,4} | T′_{4,5} | T′_{4,6} |
| T′_{5,1} | T′_{5,2} | T′_{5,3} | T′_{5,4} | T′_{5,5} | T′_{5,6} |

A 2D matrix for determining the subset of values is as follows (0 indicates un-selected values and 1 indicates selected values):

| | | | | | |
|---|---|---|---|---|---|
| 0 | 0 | 0 | 0 | 0 | 0 |
| 0 | 0 | 1 | 1 | 0 | 0 |
| 0 | 1 | 1 | 1 | 1 | 0 |
| 0 | 1 | 1 | 1 | 1 | 0 |
| 0 | 0 | 0 | 0 | 0 | 0 |

The following subset of values of the 2D matrix of temperature measurements [T_{2,3}; T_{2,4}; T_{3,2}; T_{3,3}; T_{3,4}; T_{3,5}; T_{4,2}; T_{4,3}; T_{4,4}; T_{4,5}] is compared to the following subset of values of the 2D matrix of inferred temperatures [T′_{2,3}; T′_{2,4}; T′_{3,2}; T′_{3,3}; T′_{3,4}; T′_{3,5}; T′_{4,2}; T′_{4,3}; T′_{4,4}; T′_{4,5}] by the comparison algorithm.

For illustration purposes, we choose the aforementioned first exemplary implementation of the comparison algorithm (calculation of an average of the absolute differences between the corresponding values of the two subsets). We consider that [T_{2,3}; T_{2,4}; T_{3,2}; T_{3,3}; T_{3,4}; T_{3,5}; T_{4,2}; T_{4,3}; T_{4,4}; T_{4,5}]=[38; 39; 38.5; 38; 37.5; 39; 38.5; 37; 38; 37.5] and [T′_{2,3}; T′_{2,4}; T′_{3,2}; T′_{3,3}; T′_{3,4}; T′_{3,5}; T′_{4,2}; T′_{4,3}; T′_{4,4}; T′_{4,5}]=[38; 38.5; 38; 38; 37; 39; 37.5; 38; 37; 38]. The averaged absolute difference (calculated at step 545) is equal to (|38−38|+|39−38.5|+|38.5−38|+|38−38|+|37.5−37|+|39−39|+|38.5−37.5|+|37−38|+|38−37|+|37.5−38|)/10=(0+0.5+0.5+0+0.5+0+|+|+1+0.5)/10=0.5.

If the averaged absolute difference is greater or equal than 1, the subset is declared anomalous; and the subset is declared normal if the averaged absolute difference is lower than 1 (at step 550). In the present example, the averaged absolute difference is equal to 0.5 and the subset [T_{2,3}; T_{2,4}; T_{3,2}; T_{3,3}; T_{3,4}; T_{3,5}; T_{4,2}; T_{4,3}; T_{4,4}; T_{4,5}] is declared not anomalous. If the averaged absolute difference had been equal to 1.5, the subset [T_{2,3}; T_{2,4}; T_{3,2}; T_{3,3}; T_{3,4}; T_{3,5}; T_{4,2}; T_{4,3}; T_{4,4}; T_{4,5}] would have been declared anomalous.

The preceding example is for illustration purposes only, and can be adapted to any type of comparison algorithm implemented at step 545.

Steps 515, 520, 525, 530, 540, 545 and 550 of the method 500 are performed by the control module 114 executed by the processing unit 110. Additional steps not represented in FIG. 4 may be performed by the control module 114. For example, the determination performed at step 550 is used to generate command(s) for controlling controlled appliance(s), to generate alarms, to send notifications to user(s), to display notifications on the display 150, etc.

In another implementation, steps 515-550 are performed by the IR sensor 200. The processing unit 210 of the IR sensor 200 has enough processing power to execute a neural network. The predictive model is received via the communication interface 230 of the IR sensor 200 and stored in the memory 220 of the IR sensor 200. The 2D matrix of temperature measurements is generated by the processing unit 210 of the IR sensor 200 as previously described, in relation to FIG. 2. The processing unit 210 of the IR sensor 200 executes the neural network inference engine 112, to generate the 2D matrix of inferred temperatures (step 535). The subset of values of the 2D matrix of temperature measurements is generated by the processing unit 210 of the IR sensor 200 or received from a third party via the communication interface 230 of the IR sensor 200 (step 540). The comparison algorithm is implemented by the processing unit 210 of the IR sensor 200 (step 545). The determination at step 550 is performed by the processing unit 210 of the IR sensor 200. The result of the determination at step 550 is processed internally by the IR sensor 200 or transmitted to a third party via the communication interface 230.

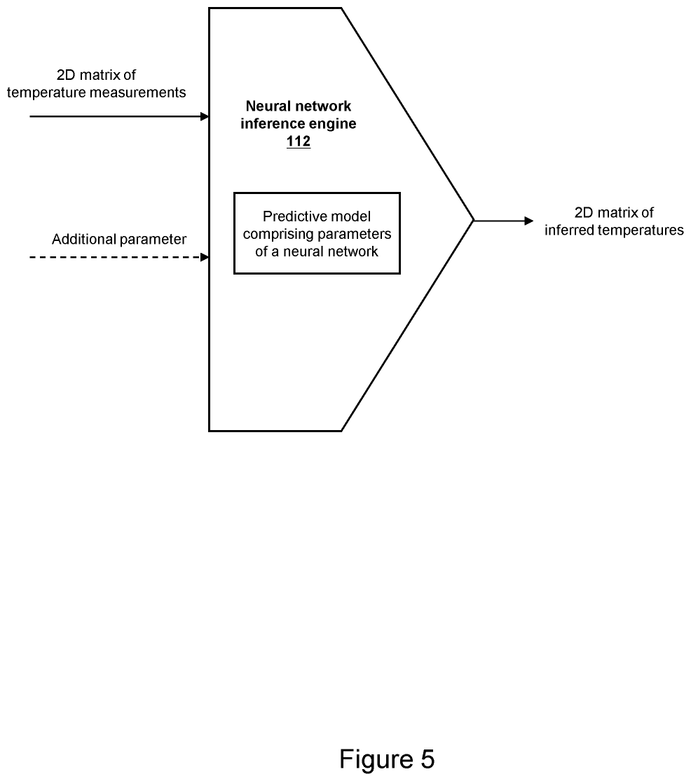

Referring to FIGS. 1, 4 and 5, a schematic representation of the neural network inference engine 112 is illustrated in FIG. 5, representing the inputs and the outputs used by the neural network inference engine 112 when performing step 535 of the method 500. For illustration purposes, the inputs include one optional additional parameter in FIG. 5. However, the inputs may include zero, one or more additional parameter(s).

Referring to FIGS. 2, 5 and 6A, an exemplary detailed representation of the neural network implemented by the neural network inference engine 112 is illustrated.

The neural network includes an input layer for receiving the 2D matrix of temperature measurements, comprising M lines and N columns, as follows:

[T_{1,1} T_{1,2} . . . T_{1,M}

T_{2,1} T_{2,2} . . . T_{2,M}

. . .

T_{N,1} T_{N,2} . . . T_{N,M}]

Each value T_{i,j} of the 2D matrix of temperature measurement is generated by the IR sensor 200, as previously detailed.

The input layer is followed by a flattening layer. As is well known in the art, the flattening layer converts the 2D matrix of temperature measurements into a one-dimensional (1D) matrix.

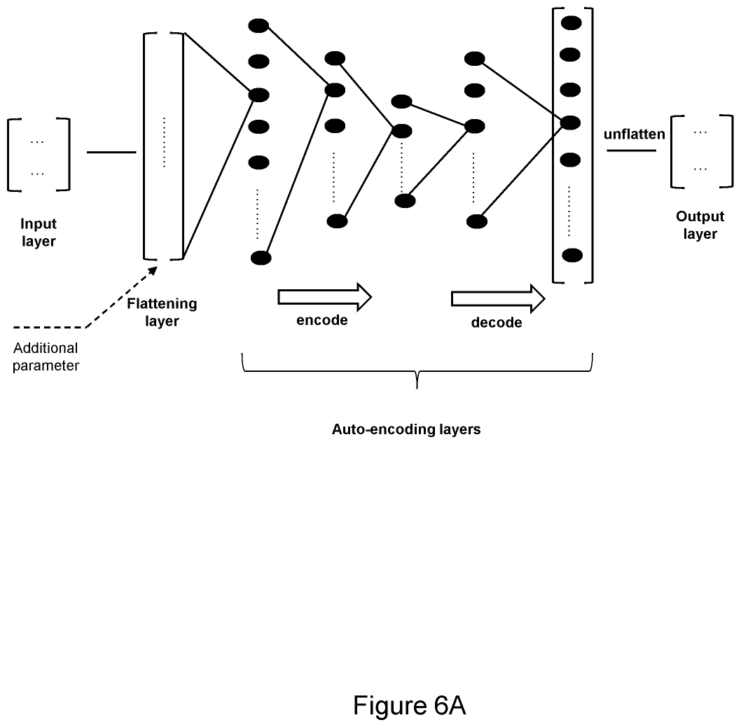

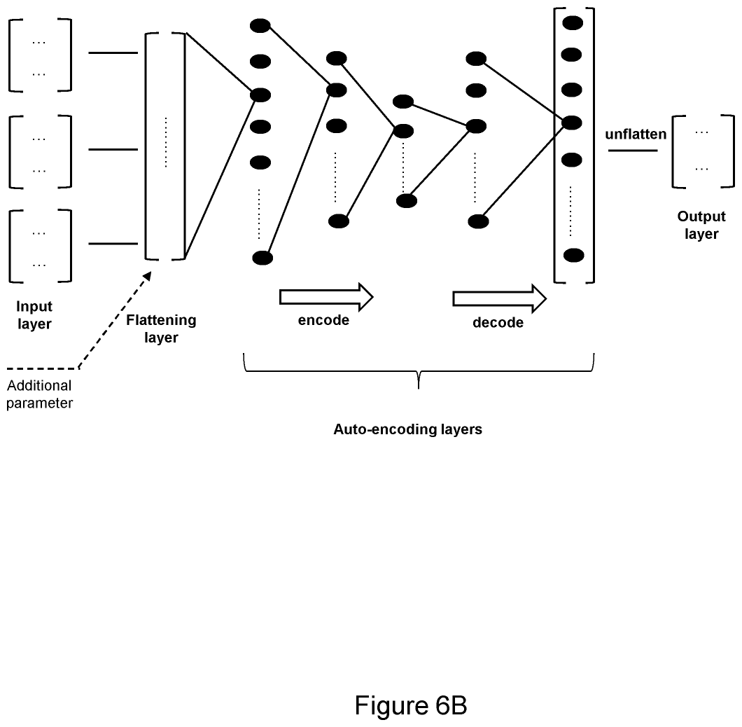

The flattening layer is followed by auto-encoding layers. The auto-encoding layers consist of fully connected layers adapted to implement the auto-encoding functionality. The number of fully connected layer(s) is an integer greater or equal than 3 for implementing the auto-encoding functionality (FIG. 6A represents five fully connected layers for illustration purposes only). The number of neurons in each fully connected layer may vary. During the training phase of the neural network, the number of fully connected layers and the number of neurons for each fully connected layer are selected, and may be adapted experimentally.

As is well known in the art, the first layer(s) among the auto-encoding layers perform an encoding functionality and the last layer(s) among the auto-encoding layers perform a decoding functionality. The central layer(s) among the auto-encoding layers has (have) less neurons than the first (encoding) and last (decoding) layers among the auto-encoding layers, to perform a compression of the data at the central layer(s).

The auto-encoding layers are followed by an output layer for outputting the 2D matrix of inferred temperatures, comprising M lines and N columns, as follows:

[T′_{1,1} T′_{1,2} . . . T′_{1,M}

T′_{2,1} T′_{2,2} . . . T′_{2,M}

. . .

T′_{N,1} T′_{N,2} . . . T′N,M]

Each value T′_{i,j} of the 2D matrix of inferred temperatures corresponds to the corresponding value T_{i,j} of the 2D matrix of temperature measurements.

As illustrated in FIG. 6A, the last layer among the auto-encoding layers is un-flattened, to convert the 1D matrix of the last layer among the auto-encoding layers into the 2D matrix of the output layer.

As illustrated in FIG. 6A, any additional parameter is injected in the flattening layer, as is well known in the art.

In another implementation not represented in the Figures for simplification purposes, the neural network represented in FIG. 6A further includes a convolution functionality. The input layer represented in FIG. 6A is followed by a two-dimension (2D) convolutional layer which generates a convoluted 2D matrix. As is well known in the art, the 2D convolutional layer is defined by the following parameters: a two-dimensions filter and a stride. The convoluted 2D matrix comprises M′ lines and N′ columns, with M greater or equal than M′ and N greater or equal than N′. As mentioned previously, any additional parameter is injected in the flattening layer and is not processed by the 2D convolutional layer.

The 2D convolutional layer is optionally followed by a pooling layer, which generates a pooled 2D matrix. As is well known in the art, the pooling layer is defined by the following parameters: a two-dimensions filter, a stride and a pooling algorithm. The pooled 2D matrix comprises M″ lines and N″ columns, with M′ greater than M″ and N′ greater than N″. The role of the pooling layer is to reduce the size of the 2D matrix generated by the convolutional layer.

The 2D convolutional layer (or the pooling layer if it is present) is followed by the flattening layer represented in FIG. 6A. The flattening layer converts the 2D matrix of the convolutional layer (or pooling layer) into a one-dimensional (1D) matrix.

Several consecutive 2D convolutional layers (and corresponding optional pooling layers) may be included in the neural network, as is well known in the art.

Referring to FIGS. 2, 5 and 6B, another exemplary detailed representation of the neural network implemented by the neural network inference engine 112 is illustrated.

The neural network includes an input layer for receiving a plurality of 2D matrices of temperature measurements. In this implementation, step 525 of the method 500 is repeated several times before performing step 535. A series of I consecutive 2D matrices of temperature measurements is received from the IR sensor 200, where I is an integer greater or equal than 2. For example, a matrix is received every second and when 3 matrices have been received (I=3), step 535 is performed (as illustrated in FIG. 6B).

The input layer is followed by a flattening layer. The flattening layer converts the I 2D matrices of temperature measurements into a 1D matrix. The flattening layer is followed by the encoding layers and the output layer, as previously illustrated in FIG. 6A. The flattening layer of FIG. 6A comprises N*M neurons, while the flattening layer of FIG. 6B comprises I*N*M neurons.

As mentioned previously, in another implementation not represented in the Figures for simplification purposes, the neural network represented in FIG. 6B further includes a convolution functionality. The input layer represented in FIG. 6B is followed by a 2D convolutional layer applying I respective 2D convolutions to the I input matrices of temperature measurements, to generate I convoluted 2D matrices.

The 2D convolutional layer is optionally followed by a pooling layer, which generates I pooled 2D matrices, respectively corresponding to the I convoluted 2D matrices of the previous layer.

The 2D convolutional layer (or the pooling layer if it is present) is followed by the flattening layer represented in FIG. 6B. The flattening layer converts the I 2D matrices of the convolutional layer (or pooling layer) into a one-dimensional (1D) matrix.

Several consecutive 2D convolutional layers (and corresponding optional pooling layers) may be included in the neural network, as is well known in the art.

A person skilled in the art would readily understand that the aforementioned implementations of the neural network are for illustration purposes only. Other implementations of the neural network may be used for achieving the functionalities of the method 500.

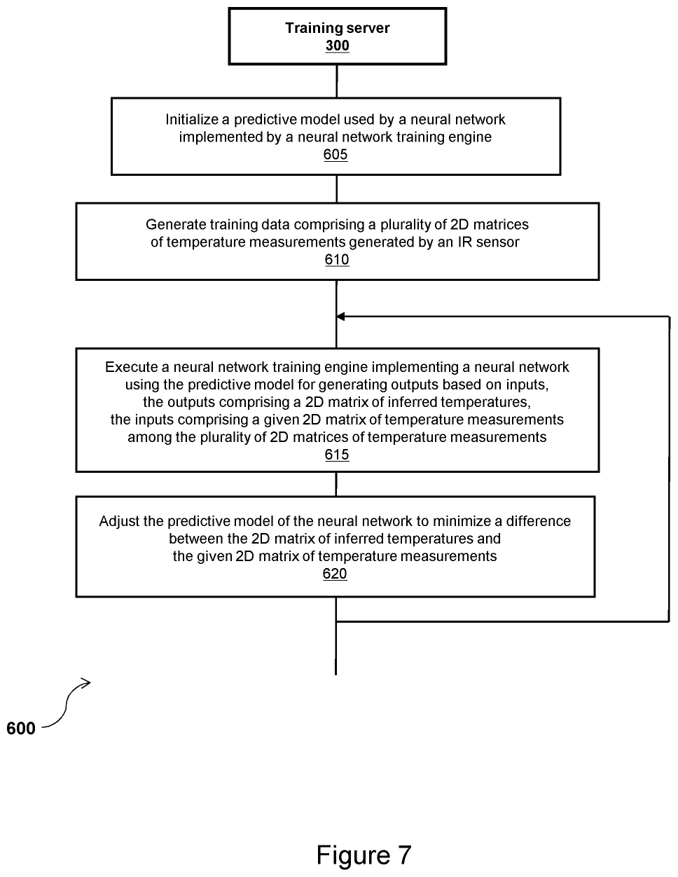

Reference is now made concurrently to FIGS. 1, 2, 3 and 7. FIG. 7 represents a method 600 for training a neural network to analyze temperature measurements of an infrared sensor. At least some of the steps of the method 600 represented in FIG. 7 are implemented by the training server 300. The present disclosure is not limited to the method 600 being implemented by the training server 300, but is applicable to any type of computing device capable of implementing the steps of the method 600.

A dedicated computer program has instructions for implementing at least some of the steps of the method 600. The instructions are comprised in a non-transitory computer program product (e.g. the memory 320) of the training server 300. The instructions provide for training a neural network to analyze temperature measurements of an infrared sensor, when executed by the processing unit 310 of the training server 300. The instructions are deliverable to the training server 300 via an electronically-readable media such as a storage media (e.g. CD-ROM, USB key, etc.), or via communication links (e.g. via a communication network through the communication interface 330).

The instructions of the dedicated computer program executed by the processing unit 310 implement the neural network training engine 311 and the control module 314. The neural network training engine 311 provides functionalities of a neural network, allowing to generate a predictive model ready to be used by the neural network inference engine 112 (executed by the computing device 100) at the end of the training, as is well known in the art. The control module 314 provides functionalities allowing the training server 300 to gather data used for the training of the neural network.

The method 600 comprises the step 605 of initializing a predictive model used by the neural network implemented by the neural network training engine 311. Step 605 is performed by the control module 314 executed by the processing unit 310 of the training server 300. The predictive model comprises various parameters which depend on the characteristics of the neural network implemented by the neural network training engine 311.

The initialization of the predictive model comprises defining a number of layers of the neural network, a functionality for each layer (e.g. convolutional layer, fully connected layer, etc.), initial values of parameters used for implementing the functionality of each layer, etc. The initialization of the predictive model is performed by a person highly skilled in the art of neural networks.

For example, the initialization of the parameters of a fully connected layer includes determining the number of neurons of the fully connected layer and determining an initial value for the weights of each neuron. Different algorithms (well documented in the art) can be used for allocating an initial value to the weights of each neuron. For example, each weight is allocated a random value within a given interval (e.g. a real number between −0.5 and +0.5), which can be adjusted if the random value is too close to a minimum value (e.g. −0.5) or too close to a maximum value (e.g. +0.5). In another example, the initialization of the parameters of a convolutional layer includes determining the size and values of the filter, and the value of the stride.

A comprehensive description of the initialization of the predictive model is out of the scope of the present disclosure, since it is well known in the art of neural networks and is different for each type of layer (e.g. a fully connected layer, a convolutional layer, a pooling layer, etc.).

The data used for the initialization of the predictive model are received from a person via the user interface 340, and/or received from a remote computing device (not represented in FIG. 3) via the communication interface 330, and/or generated by a software program executed by the processing unit 310 (e.g. random generation of the initial value of the weights).

Although not represented in FIG. 7 for simplification purposes, the predictive model initialized at step 605 is stored in the memory 320 of the training server 300.

The method 600 comprises the step 610 of generating training data. The training data comprise a plurality of 2D matrices of temperatures measurements collected by the IR sensor 200.

The IR sensor 200 is put in various operating conditions. For example, a reference object and/or reference person is submitted to various conditions. For each particular condition, the IR sensor 200 is pointed towards the reference object and/or reference person. For each particular condition, the IR sensor 200 transmits a 2D matrix of temperature measurements (related to the reference object and/or reference person), which is received by the training server 300 via the communication interface 330.

In the case where additional inputs are used by the neural network, these additional inputs are also collected at step 610 and integrated to the training data.

The method 600 comprises the step 615 of executing the neural network training engine 311. The neural network training engine 311 implements the neural network using the predictive model for generating outputs based on inputs. The execution of the neural network training engine 311 is performed by the processing unit 310 of the training server 300.

The inputs comprise a given 2D matrix of temperature measurements among the plurality of 2D matrices of temperature measurements generated at step 610. The outputs comprise a 2D matrix of inferred temperatures.

The neural network implemented by the neural network training engine 311 corresponds to the neural network implemented by the neural network inference engine 112 (same number of layers, same functionality for each layer, same parameters used for implementing the functionality of each layer, etc.). As mentioned previously, FIGS. 6A and 6B are exemplary representations of such a neural network.

The method 600 comprises the step 620 of adjusting the predictive model of the neural network, to minimize a difference between the 2D matrix of inferred temperatures (calculated at step 615) and the given 2D matrix of temperature measurements (used as inputs at step 615). Step 620 is performed by the neural network training engine 311 executed by the processing unit 310 of the training server 300.

As is well known in the art, step 620 aims at minimizing a difference between outputs calculated by the neural network and expected outputs of the neural network, through the adjustment of the predictive model of the neural network (more specifically by adjusting parameters of the neural network comprised in the predictive model). For example, for a fully connected layer of the neural network, the adjustment comprises adjusting the weights associated to the neurons of the fully connected layer. In another example, for a convolutional layer of the neural network, the adjustment comprises adjusting the values of the filter used by the convolutional layer.

As is well known in the art, steps 615-620 are repeated for several given 2D matrices of temperature measurements selected among the plurality of 2D matrices of temperature measurements (generated at step 610). The training data need to be large enough to properly train the neural network.

The implementation of the method 600 with respect to steps 610-615-620 may vary. For example, batches of N tuple(s) are selected among the training data, N being an integer greater or equal than 1. A tuple comprises a given 2D matrix of temperature measurements, and optionally the corresponding value(s) of the optional parameter(s). Step 615 is performed for the N tuple(s) of a given batch. Step 620 is performed taking into consideration the N output(s) of the neural network calculated at step 615. Thus, the adjustment of the predictive model at step 720 is performed by taking into consideration the N tuple(s) simultaneously. This process is repeated for each batch of N tuple(s). At the end of the process, a new set of batches of N tuple(s) may be selected and the process repeated.

Although not represented in FIG. 7 for simplification purposes, the modifications to the predictive model of the neural network performed at step 620 are stored in the memory 320 of the training server 300.

Steps 605, 610, 615 and 620 of the method 600 correspond to step 505 of the method 500 represented in FIG. 4.

At the end of the training phase, the neural network is considered to be properly trained (the predictive model of the neural network has been adjusted so that a difference between the expected outputs and the calculated outputs has been sufficiently minimized). The predictive model, comprising the adjusted parameters of the neural network, is transmitted to the computing device 100, as illustrated by step 510 of the method 500 represented in FIG. 4.

Test data are optionally used to validate the accuracy of the predictive model. The test data are different from the training data used at steps 610-615-620. This procedure well known in the art of neural networks is not represented in FIG. 7 for simplification purposes.

Various techniques well known in the art of neural networks can be used for performing step 620. For example, the adjustment of the predictive model of the neural network at step 620 uses back propagation. Other techniques, such as the usage of bias in addition to the weights (bias and weights are generally collectively referred to as weights in the neural network terminology), reinforcement training, etc., may also be used.

Furthermore, the evaluation of the difference between the expected outputs and the calculated outputs of the neural network at step 620 may be implemented in different ways. In an exemplary implementation, the parameters of the neural network are adjusted so that each value of the calculated 2D matrix of inferred temperatures is substantially equal to the corresponding value of the given 2D matrix of temperature measurements. A tolerance interval is usually used when performing the comparison between the calculated and expected values (e.g. a tolerance of 1% in absolute value is acceptable).

In a particular implementation, the neural network training engine 311 and the neural network inference engine 112 are implemented by the same computing device 100. In this case, the steps of the method 600 (represented in FIG. 7) and the steps of the method 500 (represented in FIG. 4) are performed by the same computing device 100 (the functionalities of the training server 300 are implemented by the computing device 100).

The training process may be implemented in two steps for a given type of IR sensor 200. The method 600 is applied to a first IR sensor of the given type, and a generic predictive model is generated. Then, for each other IR sensor of the given type, the method 600 is applied using the generic predictive model at step 605, and generating a specific predictive model adapted to each specific IR sensor of the given type. The quantity of training data for the second step is lower than the quantity of training data for the first step, since the second step consists of a fine-tuning of the training performed at the first step.

Referring now concurrently to FIGS. 4 and 7, following is a description of the application of the methods 500 and 600 to detect a medical condition (e.g. fever) of a human being (or an animal) characterized by a body temperature of the human being higher than usual.

During a training phase, the method 600 is applied to human beings with a normal body temperature. The IR sensor 200 is used to measure the body temperature of the human beings. The human beings are placed in a controlled environment, which may comprise various objects. The field of view of the IR sensor 200 includes a part of the body of the human being (e.g. his head), as well as a background comprising one or more objects (e.g. furniture, a wall, a window, plants, etc.). The 2D matrices of temperature measurements generated by the IR sensor 200 include values representative of the temperature of the part of the body of the human being, as well as values representative of the temperature of the one or more object in the background. A plurality of human beings and objects are used during the training phase, to generate a robust predictive model. At the end of the training phase, the predictive model has been adapted so that the neural network generates an output 2D matrix of inferred temperatures substantially similar to the input 2D matrix of temperature measurements, for a human being having a normal body temperature.

During an operational phase, the method 500 is applied to a given human being. The IR sensor 200 is used to measure the body temperature of the given human being. Similarly to the training phase, the 2D matrix of temperature measurements generated by the IR sensor 200 includes values representative of the temperature of the part of the body of the given human being, as well as values representative of the temperature of one or more object in the background.

The subset of values of the of the 2D matrix of temperature measurements includes at least some of the values representative of the temperature of the part of the body of the given human being. Ideally, the subset of values of the of the 2D matrix of temperature measurements includes only the values representative of the temperature of the part of the body of the given human being. The subset of values may be determined by various means, including a pattern recognition algorithm capable of detecting the body of the human being within the background.

If the given human being has a normal body temperature, the determination at step 550 is not anomalous. If the given human being has a body temperature higher than normal, the determination at step 550 is anomalous.

The comparison algorithm and the criteria (e.g. the previously mentioned thresholds) respectively used at steps 545 and 550 of the method 500 are selected and adapted during the training phase, so that the determination at step 550 is not anomalous for a human being having a normal body temperature.

Although the present disclosure has been described hereinabove by way of non-restrictive, illustrative embodiments thereof, these embodiments may be modified at will within the scope of the appended claims without departing from the spirit and nature of the present disclosure.

What is claimed is:

1. A computing device comprising:
a communication interface;
memory for storing a predictive model generated by a neural network training engine; and
a processing unit comprising one or more processor configured to:
receive via the communication interface a two-dimensional (2D) matrix of temperature measurements from an infrared (IR) sensor;
execute a neural network inference engine, the neural network inference engine implementing a neural network using the predictive model for generating outputs based on inputs, the inputs comprising the 2D matrix of temperature measurements, the outputs comprising a 2D matrix of inferred temperatures;
determine a subset of values of the 2D matrix of temperature measurements; and
apply a comparison algorithm to the subset of values of the 2D matrix of temperature measurements and a corresponding subset of values of the 2D matrix of inferred temperatures.

2. The computing device of claim 1, wherein the processing unit determines that the subset of values of the 2D matrix of temperature measurements is anomalous based on a result of the comparison algorithm.

3. The computing device of claim 1, wherein the IR sensor consists of an IR camera.

4. The computing device of claim 1, wherein the 2D matrix of temperature measurements comprises body temperature measurements of a human being, the subset of values of the 2D matrix of temperature measurements comprises at least some of the body temperature measurements of the human being, and the determination that the subset of values of the 2D matrix of temperature measurements is anomalous is indicative of the human being having a body temperature higher than usual.

5. The computing device of claim 1, wherein the neural network comprises several fully connected layers implementing an auto-encoding functionality.

6. The computing device of claim 5, wherein the neural network further comprises at least one two-dimension (2D) convolutional layer, optionally one or more pooling layer, the first among the at least one 2D convolutional layer applying a 2D convolution to the 2D matrix of temperature measurements.

7. The computing device of claim 1, wherein the processing unit receives a plurality of consecutive 2D matrices of temperature measurements from the IR sensor, and the inputs of the neural network comprise the plurality of consecutive 2D matrices of temperature measurements.

8. A method using a neural network to analyze temperature measurements of an infrared (IR) sensor, the method comprising:
storing a predictive model generated by a neural network training engine in a memory of a computing device;
receiving by the processing unit of the computing device a two-dimensional (2D) matrix of temperature measurements generated by the IR sensor;
executing by the processing unit of the computing device a neural network inference engine, the neural network inference engine implementing a neural network using the predictive model for generating outputs based on inputs, the inputs comprising the 2D matrix of temperature measurements, the outputs comprising a 2D matrix of inferred temperatures;
determining by the processing unit of the computing device a subset of values of the 2D matrix of temperature measurements; and
applying by the processing unit of the computing device a comparison algorithm to the subset of values of the 2D matrix of temperature measurements and a corresponding subset of values of the 2D matrix of inferred temperatures.

9. The method of claim 8, further comprising determining by the processing unit of the computing device that the subset of values of the 2D matrix of temperature measurements is anomalous based on a result of the comparison algorithm.

10. The method of claim 9, wherein the IR sensor consists of an IR camera.

11. The method of claim 9, wherein the 2D matrix of temperature measurements is received from the IR sensor via a communication interface of the computing device.

12. The method of claim 9, wherein the computing device consists of the IR sensor, and the 2D matrix of temperature measurements is received from an IR sensing component of the IR sensor.

13. The method of claim 9, wherein the 2D matrix of temperature measurements comprises body temperature measurements of a human being, the subset of values of the 2D matrix of temperature measurements comprises at least some of the body temperature measurements of the human being, and the determination that the subset of values of the 2D matrix of temperature measurements is anomalous is indicative of the human being having a body temperature higher than usual.

14. The method of claim 9, wherein the neural network comprises several fully connected layers implementing an auto-encoding functionality.

15. The method of claim 14, wherein the neural network further comprises at least one two-dimension (2D) convolutional layer, optionally one or more pooling layer, the first among the at least one 2D convolutional layer applying a 2D convolution to the 2D matrix of temperature measurements.

16. The method of claim 9, wherein the processing unit receives a plurality of consecutive 2D matrices of temperature measurements from the IR sensor, and the inputs of the neural network comprise the plurality of consecutive 2D matrices of temperature measurements.

17. A method for training a neural network to analyze temperature measurements of an infrared (IR) sensor, the method comprising:
(a) initializing by a processing unit of a computing device a predictive model of a neural network;
(b) generating by the processing unit of the computing device training data, the training data comprising a plurality of two-dimensional (2D) matrices of temperature measurements generated by an IR sensor;
(c) executing by the processing unit of the computing device a neural network training engine, the neural network training engine implementing the neural network using the predictive model for generating outputs based on inputs, the outputs comprising a 2D matrix of inferred temperatures, the inputs comprising a given 2D matrix of temperature measurements among the plurality of 2D matrices of temperature measurements; and
(d) adjusting by the processing unit of the computing device the predictive model of the neural network to minimize a difference between the 2D matrix of inferred temperatures and the given 2D matrix of temperature measurements.

18. The method of claim 17, wherein steps (c) and (d) are repeated for several 2D matrices of temperature measurements among the plurality of 2D matrices of temperature measurements.

19. The method of claim 17, wherein initializing the predictive model comprises determining a number of layers of the neural network, determining a functionality for each layer of the neural network, and determining initial values of parameters used for implementing the functionality of each layer of the neural network.

20. The method of claim 17, wherein the adjustment of the predictive model of the neural network uses back propagation.

21. The method of claim 17, wherein adjusting the predictive model of the neural network to minimize a difference between the 2D matrix of inferred temperatures and the given 2D matrix of temperature measurements comprises adjusting the predictive model so that each value of the 2D matrix of inferred temperatures is substantially equal to the corresponding value of the given 2D matrix of temperature measurements, optionally taking into consideration a tolerance interval.

22. The method of claim 17, wherein the neural network comprises several fully connected layers implementing an auto-encoding functionality.

23. The method of claim 22, wherein adjusting the predictive model of the neural network comprises adjusting weights of neurons of the fully connected layers implementing the auto-encoding functionality.
